(19) United States
(12) Patent Application Publication
(10) Pub. No.: US 2004/0039403 A1
(43) Pub. Date: Feb. 26, 2004

(54) Portable depilator

(76) Inventors: Dov Z. Glucksman, Wenham, MA (US); Constantine D. Pezaris, Nahant, MA (US)

Correspondence Address: George A. Herbster, 40 Beach Street, Suite 303, Manchester, MA, 01944, US

(21) Appl. No.: 10/226,526

(22) Filed: Aug. 23, 2002

(51) Int. Cl. A61B 17/50

(52) U.S. Cl. 606/133

(57) ABSTRACT

A battery-operated depilator directs current through the papilla as a low-frequency square wave. Conduction is achieved by means of tweezers or electrode patches. The electrode patches contain two conductive arrays for connecting to opposite terminals on a low-frequency AC supply receiving the alternating current independently.

### BACKGROUND OF THE INVENTION

1. Field of the Invention

This invention relates to devices for personal hygiene and more specifically to a depilator for removing unwanted hair from the body.

2. Description of Related Art

Many devices have been developed for removing unwanted hair from the body. Some devices operate mechanically by pulling the hair from the body. Others use light to effect removal. Still other devices use electrical energy for removing a single hair or multiple hairs simultaneously.

For example, United States Letters Patent No. 3,054,405 (1962) to Tapper discloses an electrical epilator or depilator in which electrical energy is applied by a needle in a pair of tweezers or forceps. The needle is one electrode of a bistable circuit. An individual grasps portions of the tweezers that are insulated from the needle thereby to establish an electrical circuit by which current passes from the needle through the papilla. In this device a 1 mA, 18 Vₚ₋ₚ square wave signal is applied to the needle a frequency of about 0.8 Hz.

United States Letters Patent No. 5,470,332 (1995) to Mehl et al. discloses a system for the permanent removal of multiple hairs as opposed to the single hair removal disclosed in the Tapper patent. The Mehl patent discloses a patch electrode with an adhesive layer, a structural layer disposed adjacent the adhesive layer, and a conductive material. The conductive material connects to a power source and contacts hairs that extend up through the adhesive layer. In use a conductive solution is applied to the skin. Then the adhesive layer is applied to the skin. Next power is applied for a period of time sufficient to destroy the matrix area of the hair, after which the hairs are removed. The power source in the Mehl patent is disclosed as a DC source, an RF source, or a source that blends DC and RF signals.

In United States Letters Patent No. 6,039,746 (2000) to Cole et al., one or more patches are secured to a skin surface to overlie multiple hairs after cleansing. A conductive gel is applied. This gel may be applied to the skin surface before applying a patch. Alternatively this gel could be incorporated in the patch. A DC current is directed between the patches to be transmitted along the hair follicles underlying the patches and through the papilla to perform an electrolysis operation. In one embodiment multiple patches are connected in parallel and the user grips a return electrode. In another embodiment some of the patches connect to a positive DC electrode and other patches connect to the negative DC electrode whereby a DC current passes between the positive and negative electrodes through the papilla under the patches.

Experience has shown that electrolysis, using a DC supply, can be painful. Depilators using RF sources tend to be complicated and therefore costly to manufacture. Moreover, these prior art systems also are not readily adapted for battery operation. What is needed is a depilator that is battery operated, that is easy to use by both professionals and individuals, that is more comfortable in use and that is adapted for a variety of applications.

### SUMMARY

Therefore, it is an object of this invention to provide an improved depilator particularly for use by individuals.

Another object of this invention is to provide an improved depilator that can be manufactured with minimal expense so it is more affordable for home use.

Still another object of this invention is to provide a battery-operated depilator.

Yet still another object of this invention is to provide a depilator that can be used in a variety of applications.

In accordance with this invention a depilator for removing hair from the body includes a power supply for generating a low-frequency signal across first and second electrical connections. A patch including first and second planar electrodes connect to the first and second electrical connections, respectively. The electrodes are disposed over discrete areas of the body in a conductive relationship. The power supply applies the low-frequency signals to the electrodes so current passes between the electrodes to interact with the papilla.

### BRIEF DESCRIPTION OF THE DRAWINGS

The appended claims particularly point out and distinctly claim the subject matter of this invention. The various objects, advantages, and novel features of this invention will be more fully apparent from a reading of the following detailed description in conjunction with the accompanying drawings in which like reference numerals refer to like parts, and in which:

FIG. 1 is a perspective view of a depilator kit in a closed configuration constructed in accordance with this invention;

FIG. 2 is a perspective view of the depilator kit in FIG. 1 in an open configuration;

FIG. 3 is an exploded view of the depilator kit shown in FIGS. 1 and 2;

FIG. 4 is an exploded view of a large electrode patch constructed in accordance with this invention;

FIG. 5 is a bottom plan view of the electrode patch shown in FIG. 4;

FIG. 6 is an edge plan view of the patch electrode shown in FIG. 4;

FIG. 7 is a bottom plan view of a small patch electrode contracted in accordance with this invention;

FIG. 8 is a bottom plan view of a shaped electrode patch constructed in accordance with this invention;

FIG. 9 is a perspective view of a tweezers electrode that is useful in accordance with this invention; and

FIG. 10 is a schematic view of a control circuit for use with each of the patch and tweezers electrodes shown in FIGS. 3 through 9.

### DESCRIPTION OF ILLUSTRATIVE EMBODIMENTS

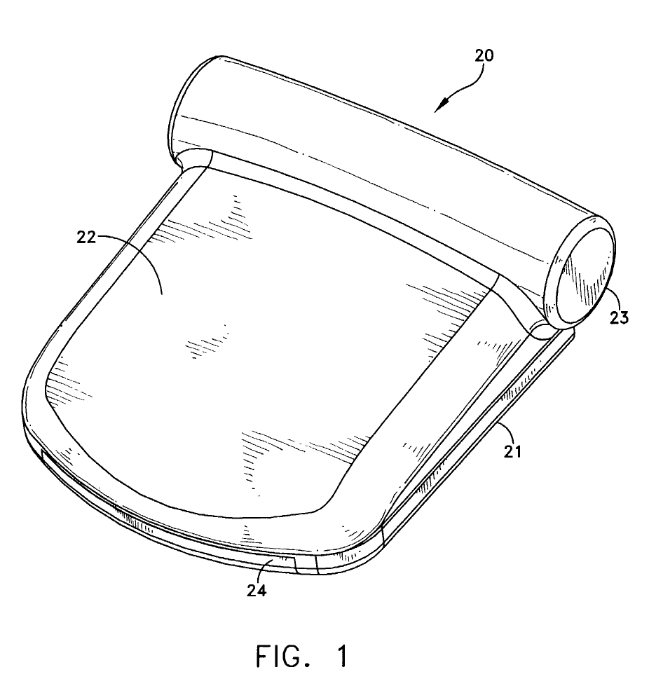

In FIGS. 1 through 3 a depilator kit 20 includes all the various components required for facilitating both single-hair and multiple-hair depilation. The depilator kit 20 includes a base 21 and a cover 22 that rotates relative to the base 21 about hinges 23 to form a case. The base 21 also supports a storage drawer 24.

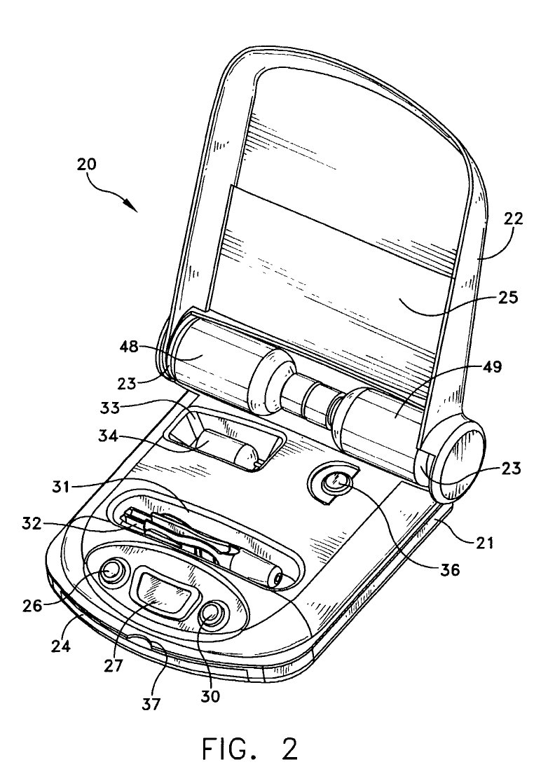

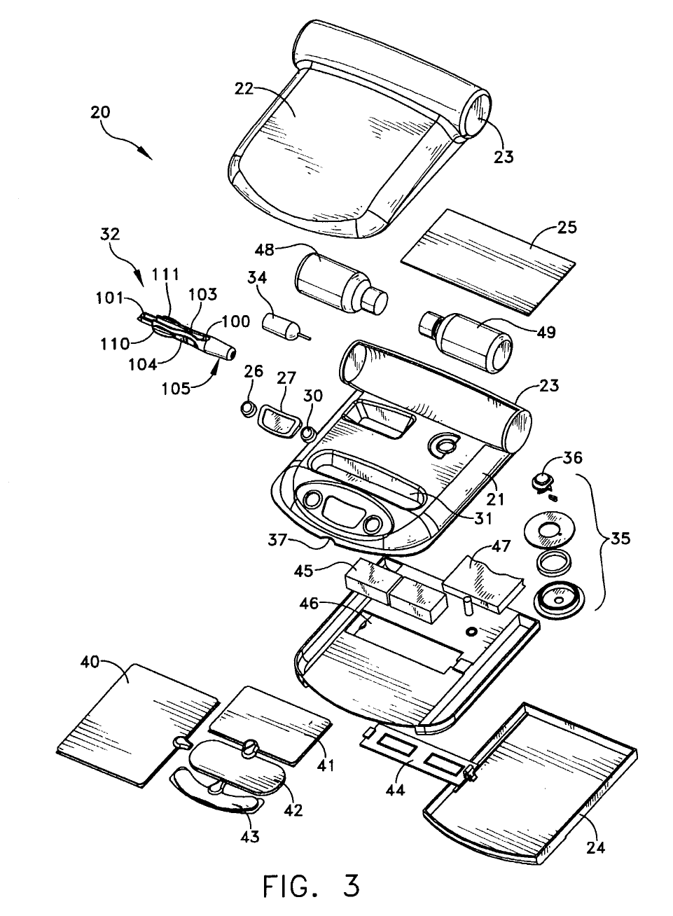

When the cover 22 opens as shown in FIGS. 2 and 3, it carries a mirror 25 for assisting in the depilation process, particularly for the removal of facial hairs. The base 21 carries an ON/OFF switch 26, an LED display 27 and a mode selector switch 30. The functions of each are described later.

A first storage compartment 31 carries tweezers 32. A second storage receptacle 33 carries an electrical adapter 34 attached to one end of an electrical cord that forms a part of a retractable cord assembly 35 shown in FIG. 3 that can be wound on a spool. A release 36 allows the spool to retract the electrical adapter 34 into the second storage receptacle 33.

Referring again to FIGS. 2 and 3 a thumb or finger notch 37 facilitates withdrawal of the storage drawer 24 from the base 21. As shown particularly in FIG. 3, the storage drawer 24 can contain one or more large electrode patches 40, intermediate electrode patches 41, small electrode patches 42, shaped electrode patches 43 or other accessories.

Referring specifically to FIG. 3 a battery cover 44 on the bottom of the base 23 provides access to batteries 45 that reside in a receptacle 46. The batteries 45 energize a controller 47, also described in greater detail later.

Again referring to FIGS. 2 and 3, the base 21 and cover 22 form a storage receptacle at the hinges 23 for two bottles. One of the bottles, such as a first bottle 48, contains a cleansing solution. The other bottle, such as a second bottle ,49, contains a conductive solution or gel.

As this depilator is battery operated, an individual can use the depilator kit 20 at any convenient location. An individual lays the depilator kit 20 on any flat surface and opens the cover 22. If the individual is going to remove a single hair, the area around the hair is cleansed with the cleansing solution from the bottle 48. Then a small amount of conductive gel from the bottle 49 is applied to be worked along the hair shaft into the hair follicle to provide a conductive path from an upper hair shaft to the papilla and surrounding area. Next the individual attaches the tweezers 32 to the electrical adapter 34 and to the hair to be removed.

To remove multiple hairs using any of the electrode patches to 41, 42, and 43, an individual cleanses the area. Then conductive gel is placed on the selected electrode patch. The patch is placed on the skin and attached to the adapter 34.

After applying the tweezers 32 or an electrode patch, the individual pushes the ON/OFF button 26 and then actuates the mode selector switch 30 until the display 27 indicates a desired treatment interval. In one embodiment, this is one of several preset intervals. When that selection is made or even as it is being made, the depilation process begins and the display 27 indicates the remaining time for the treatment. The controller 47 can visually or audibly announce the completion of the treatment interval and terminate treatment whereupon the individual can remove the electrode patch or tweezer electrodes and remove the treated hair.

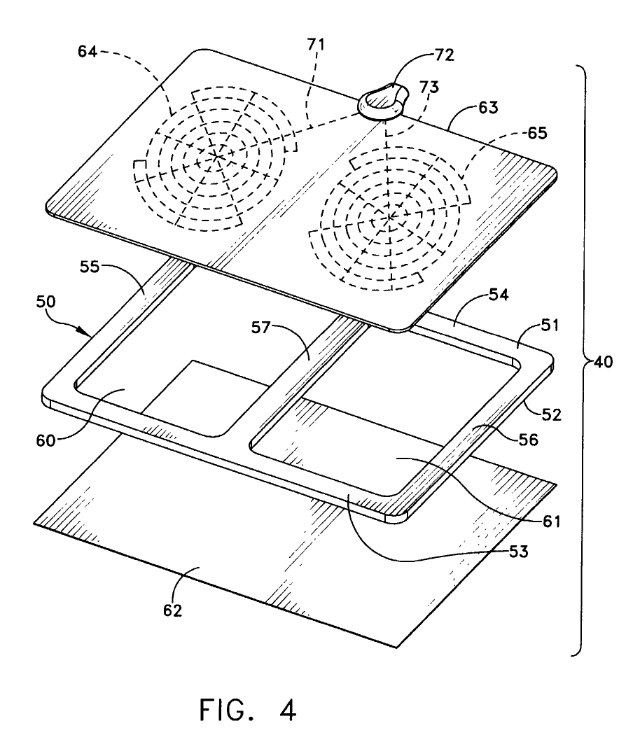

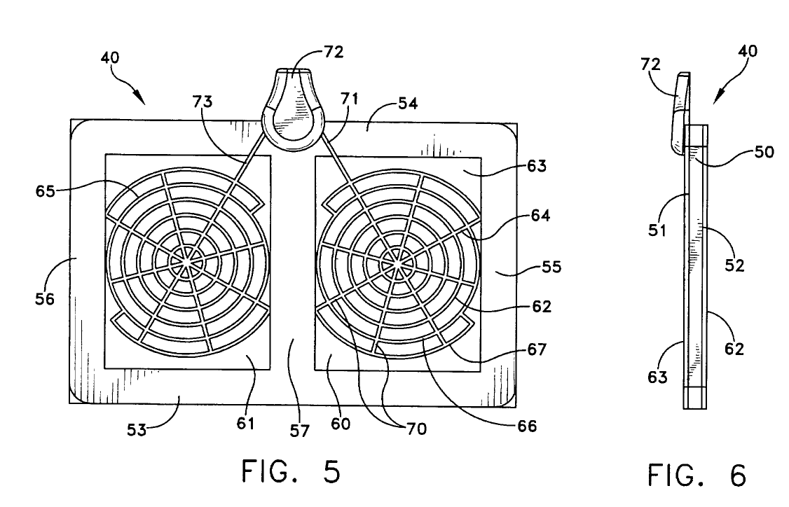

FIGS. 4 through 6 depict the large electrode patch 40. This large electrode patch 40 comprises a central planar structure 50 with adhesive-coated top and bottom sides 51 and 52. Structurally, the central planar structure 50 has elongated, parallel, spaced cross members 53 and 54, perpendicular end members 55 and 56 and a central member 57. The members 53, 54, 55 and 57 define a rectangular aperture 60; the members 53, 54, 56 and 57, a rectangular aperture 61. A thin planar protective cover 62 adheres to the bottom surface 52 to constitute a transverse cover across the apertures 60 and 61.

A contact pad 63 in the form of a thin planar structure attaches to the surface 51. This contact pad 63 has two conductive path arrays 64 and 65 centered on the apertures 60 and 61, respectively. Each of the arrays 64 and 65 has a similar structure. The array 64, by way of example, comprises spaced circular conductive paths 66 and outer concentric arcuate segments 67 with a plurality of interconnecting radial paths 70. A radial conductive path 71 connects the array 64 to a first connection on a connector 72. Another radial conductive path 73 extends from the array 65 to the second connection on the connector 72. The connector 72 provides two independent electrical paths that are insulated from each other. Also, the electrical connector 72 is adapted to connect with the electrical adapter 34. The connector 72 and adapter 34 can have any of a variety of complementary structures as will apparent to those of ordinary skill in the art. Consequently these elements are shown without any specific structure.

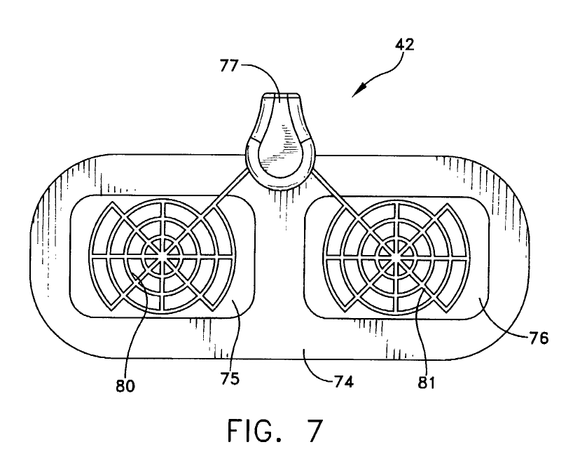

FIG. 7 is a bottom view of the small electrode patch 42. It has a similar construction to the large electrode patch 40 shown in FIGS. 4 through 6. That is, the small electrode patch 42 comprises a central support structure 74 with apertures 75 and 76 and a connector 77. Radial paths 78 connect each of the conductive path arrays 80 and 81 aligned with the apertures 75 and 76 to first and second connections in the connector 77. A protective cover, not shown, but like the protective cover 62, normally overlies the central support structure 74 to close the apertures 75 and 76 prior to use.

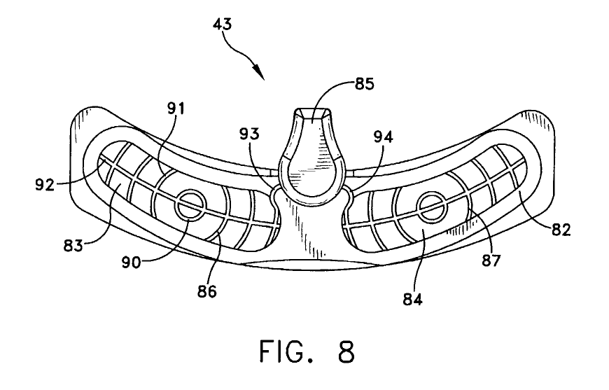

In FIG. 8 the shaped electrode patch 43 comprises a central planar structure 82 shaped for placement over the upper lip. The structure 82 has arbitrarily shaped apertures 83 and 84, and a connector 85. A conductive path array 86 aligns with the aperture 83; a conductive array 87, with the aperture 84. Both arrays are the same. In this particular electrode patch 43, the size limits the individual conduction paths to a modified form. For example, the array 86 comprises a single circumferential path 90 and a plurality of spaced concentric arcuate paths 91. A radially-extending path 92 traverses the circumferential path 90 and all the arcuate paths 91. A conductor 93 attaches to any convenient point on one of the paths and to one connection in the connector 85. A similar conductor 94 interconnects the array 87 and the connector 85.

As will be apparent, each of the electrode patches may be sized and shaped for removing multiple hairs from different parts of the body. The large electrode patches 40 might be used on the legs; the shaped electrode patch 43, over the upper lip.

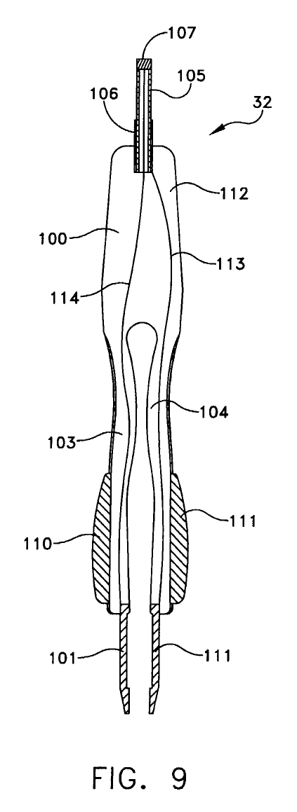

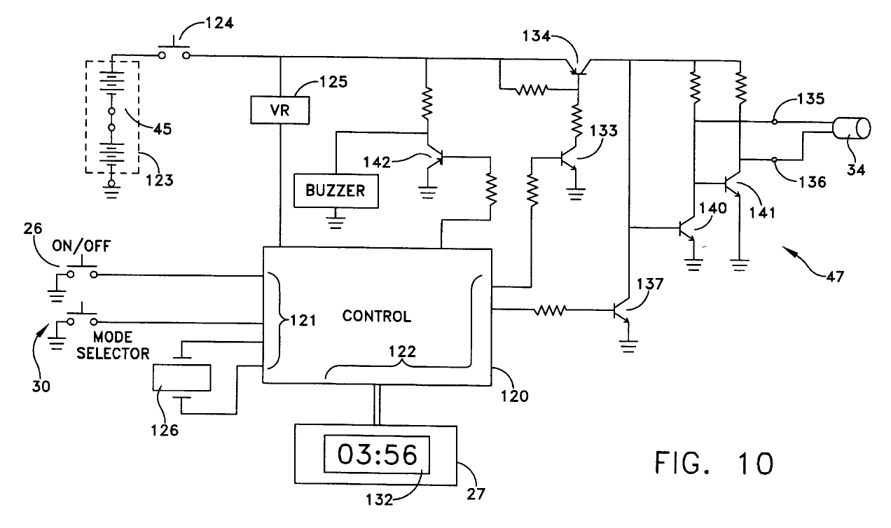

In other situations it may be desired to remove a single hair. For isolated hair removal and as previously described, the depilator 20 includes tweezers 32 shown in detail in FIG. 9. Specifically, an insulating handle 100 has conductive tweezers 101 and 102 supported by flexible bifurcated arms 103 and 104 that extend from one end of a handle 100. An adapter 105 extends from the other end of the handle 100 and contacts 106 and 107 that make electrical connection within the adapter 34. The arm 103 carries a thumb pad 110; and arm 104 carries a finger pad 111. One or both of the pads 110 and 111 has a conductive coating.

The tweezers arms 101 and 102 extend from an internal end 112. A conductor 113 is molded into the handle 100 and connects to one or both of the tweezers arms 101 and 102 and one of the contacts, such as the contact 106. Another conductor 114 molded into the handle 100 interconnects the other contact 107 and the conductive surface on the thumb pad 110 or the finger pad 111. This specific embodiment depicts connections to only the tweezers arms 101 and the thumb pad 111. However, each connection could be made to the other of the pads and/or arms, or to both pads and both arms. When the tweezers 32 of FIG. 9 are utilized, a current path includes the contact 107, the conductor 113, the conductive arm 102, the conductive gel, the papilla, the individual's body including the thumb or finger affixed to the thumb pad 110, the conductor 114 and the contact 107.

The tweezers 32 have another use as conventional tweezers. For example, after treating hair with an electrode patch, the tweezers 32 can be used to pull treated hair from the skin.

The flexibility afforded by the depilator 20 in FIGS. 1 through 3 is achieved because the controller 47 in FIG. 3 is constructed for controlling a variety of control functions. FIG. 10 depicts the controller 47 schematically. In this embodiment, the controller 47 includes a microprocessor-operated control 120 responds to signals received at different inputs 121 by generating signals at different outputs 122 in accordance with various program modules.

In this specific embodiment, two 9-volt batteries 45 connect in series to constitute an 18-volt power supply 123 connected between ground and a cover-sensing switch 124. The cover-sensing switch 124 comprises a mechanical switch, such as a reed switch or the like, that has open contacts when the cover 22 is closed. When the cover 22 opens, the switch 124 closes to energize a voltage regulator (VR) 125 that, in turn, energizes the microprocessor control 120 to establish various initial operating conditions. Thus, whenever someone closes the cover, the switch 124 isolates the power supply 123 to extend battery life.

A crystal oscillator circuit, represented by a crystal 126, provides a clock signal for the microprocessor control 120. Typically, the microprocessor control 120 includes a program module that performs a frequency divider function to obtain lower frequency signals as required. Such frequency divider program modules are known.

One of the inputs to the microprocessor control 120 is the normally open, momentarily closed ON/OFF push-button switch 26. An ON/OFF switch program module in the microprocessor 120 responds to consecutive operations of the ON/OFF switch 26 by toggling the controller 47 between an ON state and OFF state. Shifting the control to the ON state sets a variety of default and initial conditions and values. In the ON state, the microprocessor control 120 disables all outputs except for the LED display 27, which it illuminates in a default output. However, no other output functions occur. A successive actuation of the ON/OFF switch 26 shifts the control to the OFF state and blanks the LED display 27. Program modules for performing the foregoing or similar functions are known.

After an individual elects to use either the tweezers 32 or one of the electrode patches, the mode selector switch 30 is activated to establish a timer state and treatment interval. In one specific embodiment, repeated actuation of the switch 30 selects a predefined 30-second state, a 4-minute state, and an 16-minute state in sequence. As examples, a 30-second interval could be selected for the tweezers 32; a 4-minute interval, for either a small electrode patch 42 or intermediate electrode patch 44; a 16 minute interval, for either an intermediate electrode patch 41 or a large electrode patch 40. Then the individual positions the selected depilator patch with its first and second electrodes formed by the conductive path arrays. Alternatively, continued actuation of the mode selector switch could be used to select a specific patch electrode size and corresponding treatment time or to define some arbitrary treatment interval. After the mode selector switch 30 sequences to an actual number to define a treatment interval, the microprocessor control 120 sequences a display area 132 in the LED display 27 to provide a visual announcement of the minutes and seconds remaining in the treatment interval.

In addition, the microprocessor control 120 can respond to the first contact of the mode selector switch 30 by energizing a transistor 133 that shifts a power transistor 134 to a conductive state to begin a treatment interval by generating a low-frequency signal across the first and second electrodes of the tweezers or depilator patch. When the transistor 134 conducts, a potential appears on one of output connections 135 and 136 and the electrical adapter 34 with the connected electrode patch or tweezers. Concurrently, the microprocessor control 120 establishes a first switching state with a signal from one of the signal outputs 122 that biases a transistor 137 to a non-conductive state. The collector voltage on the transistor 137 establishes a base-emitter voltage on a transistor 140 that shifts the transistor 140 into a conductive state and simultaneously shifts a cascaded transistor 141 to a non-conducting state. Consequently, in this mode the output connection 135 is substantially at a ground potential while the output connection 136 is at the potential established by the power supply 123, essentially 18 volts with the 18-volt power supply 123.

The microprocessor 120 includes a power switching program module that shifts the switching transistor 137 between the conductive and non conductive states at a low frequency, e.g. 1 Hz or less by generating a low frequency control signal. After one-half second or other interval for other frequencies, the microprocessor control 120 shifts the low frequency control signal, so the transistor 137 shifts to a conducting state. This turns the switching transistor 140 off, and the switching transistor 141 conducts. Now the terminal 135 is at the positive power supply potential and the output connection 136 is at ground potential. Consequently, so long as the microprocessor 120 performs this frequency function, an essentially 1 Hz or other low-frequency square wave appears across the output connections 135 and 136. The implementation of such a power switching program module is well within the capabilities of persons of ordinary skill in the art.

When any of the selected intervals expire, the microprocessor 120 shifts the switching transistor 133 to a non-conductive state, thereby shifting the transistor 134 to a non-conductive state and de-energizing the voltage across the output connections 135 and 136. At the same time, the display area 132 shifts to a zero value, thereby indicating the completion of the treatment.

The circuit shown in FIG. 10 also includes a switching transistor 142. The microprocessor control 120 uses this transistor 142 to announce the end of a treatment interval. For example, the microprocessor control 120 could incorporate an annunciator program function to switch the transistor 142 at an audio frequency for some predetermined interval after the treatment ends thereby to produce an audible announciation through some electro-acoustic transducer, such as a loudspeaker. Alternatively, this program function could keep the transistor 142 in a non-conductive state except for a short interval at the end of a treatment whereupon terminating conduction through the transistor 142 would apply an operating potential to a conventional buzzer circuit 143.

As will now be apparent the depilator kit 20 in FIGS. 1 through 3 can be operated to remove a single hair or multiple hairs spread over an area using optional treatment intervals. For removing a single hair, an individual washes the area around that hair and treats the skin at that hair with the solutions from the bottles 48 and 49, respectively. Then the individual attaches the tweezers 32 to the electrical adapter 34 and actuates the ON/OFF switch 26. The individual can use one hand to capture the selected single hair in the ends of the tweezers 32 by gripping it tightly and use the other hand to actuate the mode selector switch 30 to select an appropriate interval, typically 30 seconds, as previously indicated. The display area 132 visually indicates the remaining time for the treatment.

If the individual desires to use one of the electrode patches, the individual uses the cleaning fluid in the bottle 48 to wash an area of skin to be treated. Then a protective cover on a selected electrode patch, such as the protective cover 62 in FIG. 4, can be removed. Conductive gel from the bottle 49 is placed in the cavities defined by the apertures, such as apertures 60 and 61 in the large electrode patch 40. Then the selected electrode patch is applied to the cleansed skin area. The contact pad, such as the contact pad 63 in FIGS. 4 through 6, can be pressed to distribute the conductive gel throughout the areas that are coextensive with the apertures.

Next the individual sequences the mode selector switch 30 until the microprocessor 120 displays the appropriate treatment interval. During the treatment interval, the low-frequency signal establishes a path from one conductive array to the other through the conductive gels and papilla under both electrode patches. When the treatment interval ends, the electrode patches can be removed from the skin. The tweezers 32 then can be used conventionally to remove each individual hair that was treated to kill the roots by galvanic action.

Therefore, in accordance with the various objects of this invention, a depilator has been disclosed that is battery operated, self-contained, portable, and easily used. The low-frequency alternating current energization reduces painful sensations that can otherwise occur during such operations with DC.

The structures shown in the various figures depict but one specific embodiment for implementing this invention. Many other variations are possible. Different conductive arrays, variations on the various electrode structures and different control sequences can all be implemented by those of ordinary skill in the art.

Therefore, it is the intent of the appended claims to cover all such variations and modifications as come within the true spirit and scope of this invention.

What is claimed as new and desired to be secured by Letters Patent of the United States is:

1. A depilator for removing hair from discrete areas of the body comprising:
A) a power supply for generating a low frequency signal across first and second electrical connections, and
b) a patch including first and second spaced planar electrodes connected to said first and second electrical connections, respectively, said patch disposing said electrodes over discrete areas of the body in a conductive relationship therewith whereby said power supply causes a low-frequency signal to pass between said planar electrodes and to interact with the papilla:

2. A depilator as recited in claim 1 wherein said patch includes a central planar structure with first and second apertures therethrough, a contact pad with said first and second electrodes attached to one side of said central planar structure and a removable sheet attached to the other side of said planar structure.

3. A depilator as recited in claim 2 wherein each of said first and second electrodes defines an array of equipotential conductive paths formed on a surface of said contact pad juxtaposed said central planar support, each said array being positioned over one of said apertures.

4. A depilator as recited in claim 3 wherein each of said arrays includes angularly spaced radially conductive paths interconnected with radially-spaced circumferential conductive paths.

5. A depilator as recited in claim 4 wherein said first electrical connection connects to one of said radial conductive paths in said array constituting said first electrode and said second electrical connection connects to one of said radial conductive paths in said array constituting said second electrode.

6. A depilator as recited in claim 2 wherein said central planar support apertures, said contact pad and said back sheet define first and second cavities and wherein said path includes a conductive gel in each of said cavities, said conductive gel coming into contact with the skin when said back sheet is removed from said central planar support.

7. A depilator as recited in claim 2 wherein said central planar support is formed of a foam material with adhesive material on each of the planar surfaces, said adhesive material on one planar surface contacting said contact pad and said adhesive surface on the other planar surface contacting said back sheet and contacting the skin when said back sheet is removed thereby to stabilize the position of the patch on the skin.

8. A depilator as recited in claim 7 wherein said central planar support apertures, said contact pad and said back sheet define first and second cavities and wherein said path includes a conductive gel in each of said cavities, said conductive gel coming into contact with the skin when said back sheet is removed from said central planar support.

9. A depilator as recited in claim 1 wherein said power supply includes a battery, a low frequency switching control and a switching circuit responsive to said switching control and connected between said battery and said first and second electrical connections thereby to apply an alternating current potential across said first and second electrodes.

10. A depilator as recited in claim 9 wherein said switching control includes means for establishing a switching frequency of 1 Hz.

11. A method for removing hair from a discrete area of the body by means of a depilator comprising:
A) positioning a patch associated with the depilator including first and second spaced, electrically isolated planar electrodes on the discrete area,
B) defining a treatment interval, and
C) generating a low frequency signal across first and second planar electrodes for the treatment interval.

12. A method as recited in claim 11 wherein said generation of the low frequency signal includes providing a battery voltage, generating a low frequency control signal for the treatment interval and switching the polarity of the voltage across the electrodes whereby a low-frequency alternating current signal is applied to the hairs in contact with the electrodes.

13. A method as recited in claim 12 for use with a selected patch taken from a group of differently sized patches, wherein said method includes defining a different treatment interval for each differently sized patch and selecting that treatment interval for treatment.

14. A method as recited in claim 12 comprising the step of announcing the end of a treatment interval.

15. A method as recited in claim 12 comprising the step of displaying the time remaining in the treatment interval.

16. A method as recited in claim 12 wherein said low frequency signal has a frequency of about 1.0 Hz.

17. A method as recited in claim 12 additionally comprising the step of isolating the battery when the depilator is inactive.
